(19) United States
(12) Patent Application Publication
(10) Pub. No.: US 2008/0219940 A1
(43) Pub. Date: Sep. 11, 2008

(54) Colored cosmetic composition having soft focus effect

(75) Inventors: Gaetan Chevalier, Boigny Sur Bionne (FR); Franck Girier-Dufournier, Paris (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/153,236

(22) Filed: May 15, 2008

Related U.S. Application Data
(63) Continuation of application No. 10/902,185, filed on Jul. 30, 2004, now abandoned.

(30) Foreign Application Priority Data
Aug. 1, 2003 (FR) 03 09572

(51) Int. Cl. A61K 8/18; A61Q 1/02

(52) U.S. Cl. 424/63

(57) ABSTRACT

Colored cosmetic compositions include, in a physiologically acceptable medium, a filler and a coloring agent, the coloring agent having a transparency and/or being present in a quantity such that presence of the coloring agent with the filler in the composition confers a saturation C* of greater than or equal to 17, a hemispherical transmittance greater of than or equal to 70% and a soft focus index H of greater than or equal to 40%.

### CROSS-REFERENCE TO RELATED APPLICATION

This is a Continuation of application Ser. No. 10/902,185 filed Jul. 30, 2004. The disclosure of the prior application is hereby incorporated by reference herein in its entirety.

This application claims the benefit of French Application No. 03 09572 filed on Aug. 1, 2003, the entire disclosure of which is incorporated by reference herein.

### BACKGROUND OF THE INVENTION

The present invention relates to cosmetic compositions for treating surfaces such as skin, mucous membranes and nails.

There exists a need to benefit from a composition capable of providing color and hiding defects, for example for the skin, while enabling a treated surface to retain a natural appearance.

### SUMMARY OF THE INVENTION

In various exemplary embodiments, the present invention provides colored cosmetic compositions comprising, in a physiologically acceptable medium, a filler and a coloring agent, the coloring agent having transparency and/or being in a quantity such that its presence with the filler in the composition confers a saturation C* of greater than or equal to 17, hemispherical transmittance of greater than or equal to 70% and a soft focus index H of greater than or equal to 40%.

Exemplary compositions, for example when applied to the skin, can enable imperfections to be masked and can provide color without rendering the makeup particularly visible. A relatively natural appearance of a made-up surface can thus be conserved.

For a better understanding of the invention as well as other aspects and further features thereof, reference is made to the following drawings and descriptions.

### BRIEF DESCRIPTION OF THE DRAWINGS

Various exemplary embodiments of the invention will be described in detail with reference to the following figures, wherein:

FIG. 1 is a representation of hemispheric spectral transmission Th(λ) of a composition on a quartz slide;

FIG. 2 is a representation of direct spectral transmission Td(λ) of a composition on a quartz slide; and

FIG. 3 is graph depicting an exemplary range V for values of hemispherical transmittance Th, soft focus index H, and saturation C* for compositions according to this invention.

### DETAILED DESCRIPTION OF EXEMPLARY EMBODIMENTS

### Measuring Hemispherical Transmittance Th and Calculating Soft Focus Index H

In various exemplary embodiments, cosmetic compositions according to this invention have a soft focus effect H that is greater than or equal to 40%, greater than or equal to 50%, greater than or equal to 60%, greater than or equal to 70% or greater than or equal to 80%.

In various exemplary embodiments, hemispherical transmittance Th is greater than or equal to 70%, greater than or equal to 80% or greater than or equal to 85%.

The term “soft focus index H” refers to magnitudes ((Th−Td/Th)×100 where Th represents hemispherical transmittance and Td represents a direct transmission factor.

When soft focus index H is high, a composition produces a large soft focus effect, serving to mask defects by changing the perception of relief.

Hemispherical transmittance Th provides information about transparency of a composition. The greater the value of Th, the more transparent a composition is.

Th and Td can be measured using a spectrophotometer and an integration sphere placed behind a composition that is to be characterized.

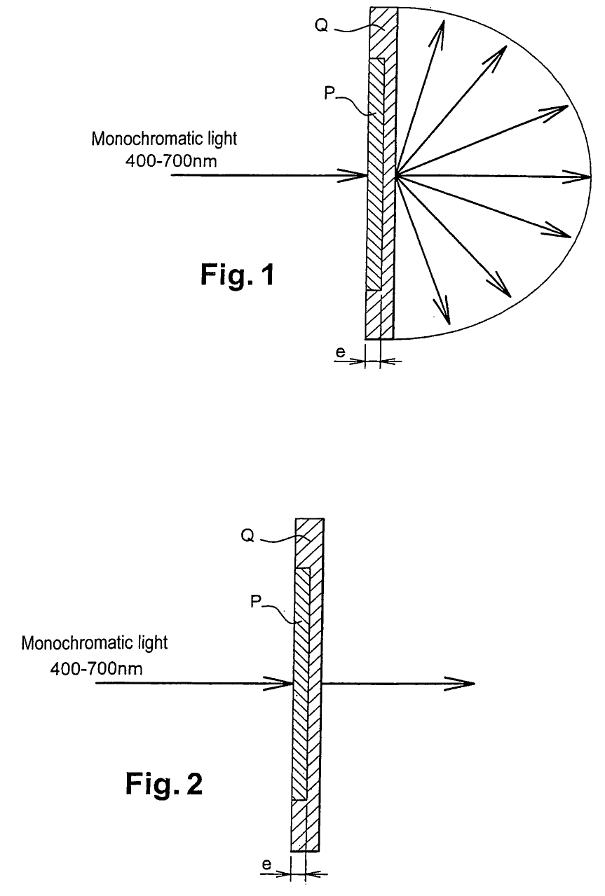

The hemispherical spectral transmittance Th(λ) of a composition is defined by a ratio of intensity of light of wavelength λ received by a sample of composition P to intensity of light delivered by the sample in all directions in a space defined by a plane, as shown in FIG. 1.

Direct spectral transmittance Td(λ) of a sample is defined by a ratio of intensity of light of wavelength λ received by a sample to intensity of light delivered by the sample in the same direction of propagation as incident light, as shown in FIG. 2.

Hemispherical and direct spectral transmittance Th(λ) and Td(λ) can be measured, for example, using the following experimental protocol.

A composition P for analysis is spread over an indented quartz slide Q so as to form a layer of thickness e of 20 micrometers (μm) and is then placed in an oven for 5 minutes (min) at 37° C.

Th(λ) can be measured using a Varian Cary 300® spectrophotometer and an integration sphere of trademark Labsphére® placed behind a quartz slide containing a composition. A spectrophotometer is used in diffuse transmission mode and wavelength λ of monochromatic incident light varies over a range of 400 nanometers (nm) to 700 nm.

Measurement can be performed in % T transmission mode at a scanning rate of 240 nm/min, and in “double-reverse” mode.

Initially, calibration is performed by taking a first measurement on a quartz slide Q while empty in order to obtain a maximum value of transmitted light intensity.

Thereafter, hemispherical spectral transmittance Th(λ) is measured using a quartz slide containing composition P that is to be analyzed.

Td(λ) is measured by means of a spectrophotometer as described above, used in direct transmission mode, and the wavelength λ of the incident monochromatic light is varied over a range of from 400 nm to 700 nm. Measurement is performed in % T transmission mode with scanning at a rate of 240 nm/min, and in “double” mode. An empty quartz slide is placed in a reference compartment, and a quartz slide containing the composition is placed in a measurement compartment, and then Td(λ) is measured.

Hemispherical and direct transmission factors Th and Td can be calculated by establishing the respective means of all of values Th(λ) and Td(λ) obtained while varying wavelength over a range of from 400 nm to 700 nm.

### Saturation C*

Saturation C* can be measured, for example, in CIE's L*a*b*C*h color space as follows.

A composition is deposited flush with a galvanized steel cup possessing a depth of at least 2.47 millimeters (mm).

A cup is then covered by an H1 type “LMR” object-carrier slide with chamfered edges, having dimensions of 76 mm×26 mm, from Labo-Moderne.

Saturation C* can be measured by means of a CM3700d spectrocolorimeter, lighting and observation systems being in d/8° reflectance mode, and measurements performed in specular reflection mode being excluded. Ultraviolet (UV) emission is included 100%. Zoom position is mean (MAV), aperture is mean, configuration is CREIMM, observation is 10° CIE 1964 and illuminant is D65.

In various exemplary embodiments, saturation C* of a composition, measured, for example, by the above technique, is greater than or equal to 17 or greater than or equal to 20. In various exemplary embodiments, saturation C* of a composition is less than or equal to 60.

A value greater than 17 makes it possible to obtain sufficient color contribution, while a value of less than 60 makes it possible to avoid harming a soft focus effect and to conserve natural-looking makeup, as appropriate.

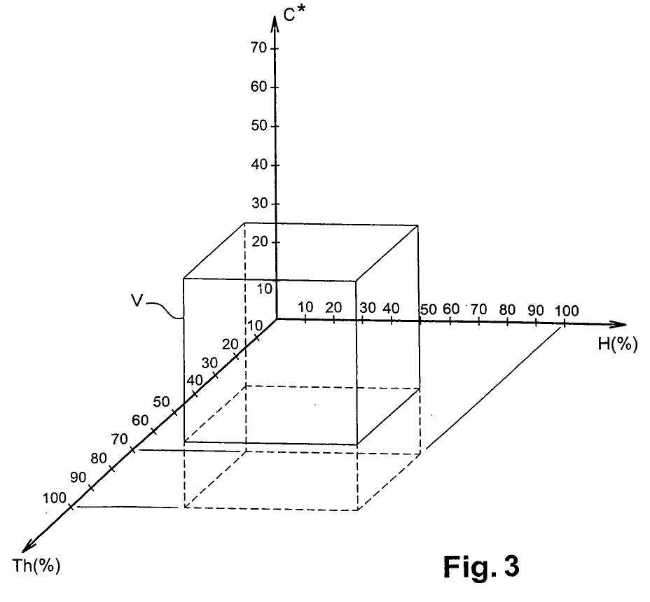

FIG. 3 shows an exemplary range V for values Th, H, and C* of a composition in Th, H, and C* space.

### Coloring Agent

As used herein, the term “coloring agent” refers to any pigment or dye or mixture of pigments and/or dyes suitable for providing sufficient color to a composition to enable it to present a desired saturation C*, while also making it possible to obtain desired values for soft focus index H and for hemispherical transmittance Th.

In various exemplary embodiments, a coloring agent may comprise or be constituted by particles of at least one pigment. Particles of pigment may have been subjected, where appropriate, to treatment to increase color stability and facilitate incorporation of particles in a composition. For example, pigment particles treated to make them hydrophobic are easier to disperse in an oily phase.

Pigment particles may have a variety of shapes, for example they may be substantially spherical in shape or they may be flat.

Pigment particles may have a multilayer structure, for example a transparent core made of, e.g., silica.

Transparent-core pigment particles serve to avoid making a composition excessively opaque, which is favorable for obtaining desired values of H and Th.

Pigments may be non-interference pigments and/or non-fluorescent pigments.

In pigments including particles having a relatively transparent single-layer structure, the quantity of pigment can be relatively high, for example greater than or equal to 1% by weight, in a range of from 1.5% to 10% by weight or in a range of from 2% to 8% by weight, relative to a total weight of a composition. In various exemplary embodiments, pigment content can be in a range of from 2% to 5%, for example about 3%.

The higher the transparency of a pigment, the greater the quantity of pigment that could, a priori, be incorporated without causing a composition to lose desired soft focus effect and hemispherical transmission factor.

Transparency of a pigment can be quantified relative to contrast, for example as defined below, and for pigments as defined above, a contrast ratio may be, for example, in a range of from 15 to 65 or less than or equal to 50. Further, contrast ratio can be, for example, less than or equal to 45, less than or equal to 40 or less than 35.

Coloring agents may comprise pigments having a structure which can be, for example, of the sericite/brown iron oxide/titanium dioxide/silica type. Such pigments are sold, for example, under the reference Coverleaf NS or JS by Chemicals and Catalysts, and present a contrast ratio of close to 30.

Coloring agents may also comprise pigments having a structure which may, for example, be of the silica microsphere type containing iron oxide. An example of a pigment presenting such a structure and having a relatively low contrast ratio is that sold by the supplier Miyoshi under the reference PC BALL PC-LL-100 P, which pigment is constituted by silica microspheres containing yellow iron oxide.

Relatively small values for contrast ratio, combined with soft focus index and hemispherical transmittance values, make it possible to obtain results that are particularly satisfactory in terms of combined delivery of color and covering power, while maintaining a large soft focus effect to mask imperfections.

When using a mixture of pigments, proportions of pigments may be adjusted as a function of their contrast ratios.

Exemplary compositions may be free from pigments having contrast ratios greater than 60.

In various exemplary embodiments, compositions may also include particles of at least one pigment that is relatively opaque, for example having a contrast ratio greater than 60, provided that a quantity of such pigment introduced into a composition does not impede obtaining desired values of H and Th.

In various exemplary embodiments, compositions may include conventional pigments having a relatively high contrast ratio, e.g., greater than or equal to 40, such as pigments of iron oxides and/or titanium dioxide type.

Exemplary pigments may have a core that is not transparent.

Exemplary compositions may include less than 2%, less than 1.5%, less than 1% or less than 0.75% by weight of such relatively opaque pigments. In various exemplary embodiments, compositions may include from 0.25% to 0.75%, for example about 0.5%, of such relatively opaque pigments relative to a total weight of a composition.

Coloring agents may also be selected from dyes, such as hydrosoluble or liposoluble dyes, or still other coloring agents, provided that their incorporation in a composition does not impede obtaining desired soft focus index H and hemispherical transmittance Th values. Coloring agents may thus comprise a hydrosoluble dye such as, for example, caramel.

Coloring agents may be of a color that corresponds to that of skin to which a composition is to be applied. For example, coloring agents may include at least one brown, yellow or red pigment.

In various exemplary embodiments, coloring agents may be free from pearlescent agents.

### Measuring Contrast Ratio

Contrast ratio may be defined as set forth in WO 98/52534, which is incorporated herein by reference in its entirety.

In order to calculate contrast ratio, the following procedure may be performed.

A mixture is formed comprising 5% by weight relative to a total weight of a coloring agent under study with the remainder being the following reference emulsion, which mixture is applied as a film having a thickness of 50 μm both on a black opaque medium and on a white opaque medium. The film is dried for 24 hours (h) at 25° C. ±1° C. under a pressure of 1 atmosphere (atm).

The reference emulsion has the following formulation:

TABLE 1

| | % by weight |
|---|---|
| Water | 45.83 |
| Methyl p-hydroxybenzoate | 0.45 |
| Chlorphenesine | 0.34 |
| Disodium EDTA | 0.11 |
| Glycerin | 5.62 |
| PEG-8 | 2.25 |
| PEG-20 | 1.12 |
| Magnesium and aluminum silicate | 0.9 |
| Sodium lauryl sarcosinate | 1.68 |
| Titanium dioxide (and) alumina (and) | 3.37 |
| glycerin (and) silica | |
| Triethanolamine | 1.3 |
| Steric acid | 2.7 |
| Glyceryl stearate | 2.02 |
| Butyl p-hydroxybenzoate | 0.17 |
| Isononyl isononanoate | 8.99 |
| Cyclohexsiloxane | 6.57 |
| Dimethicone | 10.28 |
| BIS-PEG-15 methyl ether | 2.25 |
| dimethicone | |
| Talc | 1.12 |
| Kaolin | 1.12 |
| Polymethyl methacrylate | 1.69 |

Using a colorimeter, e.g., of the Minolta CR-200 type, in D65 illuminant mode, with a viewing angle of 0°, a tristimulus value Y of a composition is measured at three different locations on a black medium and at three different locations on a white medium.

Contrast ratio corresponds to a mean of three Y values measured on a black medium divided by a mean of three Y values measured on a white medium, multiplied by 100.

The higher the contrast ratio, i.e., the closer to 100%, the more a coloring agent is opaque. The lower the contrast ratio, the more a coloring agent is transparent.

### Fillers

Exemplary fillers may be of any chemical kind provided that they are compatible for use in a cosmetic and so long as they do not interfere with desired properties of compositions according to this invention.

Exemplary compositions according to this invention include at least one filler referred to as a soft focus effect filler, contributing to giving a composition, in the presence of a coloring agent, a desired soft focus index H and hemispherical transmittance Th.

Soft focus effect fillers may be substantially colorless within a composition.

Soft focus effect fillers suitable for use in the composition of the invention may, for example, have a number mean size of less than or equal to 15 μm, less than or equal to 10 μm or less than or equal to 7.5 μm. For example, soft focus effect fillers may have a number mean size of from about 1 μm to 5 μm.

The term “number mean size” refers to a dimension given by a half-population statistical grain size distribution, referred to as D50.

Soft focus effect fillers used may include, but are not limited to, powders of silica and silicates, alumina, powders of polymethyl methacrylate, talc, silica/TiO₂ or silica/zinc oxide composites, polyethylene powders, starch powders, polyamide powders, powders of styrene/acrylic copolymers, silicone elastomers and mixtures thereof.

Examples of soft focus effect fillers include, but are not limited to:

talc having a mean size less than or equal to 3 μm, in particular the talc sold under the trade name Talc P30 by the supplier Nippon Talc;

Nylon® 12 powder, in particular that sold under the name Orgasol 2002 Extra D Nat Cos® by the supplier Atochem;

microbeads of silica such as those sold under the name SB-700® or SB-150® by the supplier Miyoshi;

microspheres of amorphous silica, for example those referenced Sunsphere H-53, having number mean size of 5 μm, by the supplier Asahi Glass; and

silica powders with mineral wax surface treatment (1% to 2%) from the supplier Degussa.

Soft focus effect fillers may be present in cosmetic compositions according to this invention at a content of from 0.1% to 20% by weight, from 1% to 12% by weight, from 5% to 10% by weight relative to a total weight of a composition. For example, soft focus effect fillers may be present at a content of about 8% by weight relative to a total weight of a composition. In various exemplary embodiments, content of soft focus effect fillers may be selected, for example, as a function of a nature and/or a quantity of coloring agent and as a function of a desired effect.

### Physiologically Acceptable Medium

Exemplary compositions according to this invention comprise a physiologically acceptable medium.

The term “physiologically acceptable medium” refers to a medium that is not toxic and that is suitable for application to skin, for example to the face and/or the neck, on mucous membranes such as the lips or on keratinous surfaces such as the fingernails of human beings. Physiologically acceptable media are generally adapted to the nature of a medium on which a composition is to be applied, and also to a form in which a composition is packaged.

Exemplary compositions according to this invention may be emulsions. A proportion of a fatty phase in a composition may be, for example, from 5% to 80% by weight or from 5% to 50% by weight relative to a total weight of a composition.

In various exemplary embodiments, compositions may comprise an aqueous medium constituting an aqueous phase forming a continuous phase of a composition.

An aqueous phase may be constituted essentially by water. Nevertheless, an aqueous phase may also comprise a mixture of water and an organic solvent that is miscible in water, for example an organic solvent having miscibility in water greater than 50% by weight at 25° C., such as lower monoalcohols having 1 to 5 carbon atoms such as, for example, ethanol, isopropanal, glycols having 2 to 8 carbon atoms such as propylene glycol, ethylene glycol, 1,3-butylene glycol, dipropylene glycol, C₃-C₄ ketones and C₂-C₄ aldehydes.

Aqueous phases comprising in particular water and possibly an organic solvent that is miscible in water, may be present in a composition at a content of from 1% to 95% by weight, from 3% to 80% or from 5% to 60% by weight, relative to a total weight of a composition.

In various exemplary embodiments, compositions may also comprise a fatty phase.

A fatty phase of a composition may comprise, for example, at least one oil, for example an oil selected from:

hydrocarbon oils of animal origin, such as perhydrosqualene;

hydrocarbon oils of vegetable origin, such as liquid triglycerides of fatty acids having 4 to 10 carbon atoms and the liquid fraction of karite butter;

synthetic esters and ethers, in particular of fatty acids, such as oils having formulae R¹CORR² and R¹OR² in which R¹ represents the remainder of a fatty acid having 8 to 29 carbon atoms, R² represent an optionally branching hydrocarbon chain containing 3 to 30 carbon atoms, such as for example Purcellin oil, isononyl isononanoate, isopropyl myristate, ethyl-2-hexyl palmitate, octyl-2-dodecyl stearate, octyl-2-dodecyl erucate, isostearyl isostearate; hydroxyl esters such as isostearyl lactate, octylhydroxystearate, octyldodecyl hydroxstearate, diisostearyl-malate, triisocetyl citrate, heptanoates, octanoates, decanoates of fatty acids; polyol esters such as propylene glycol dioctanoate, neopentylglycol diheptanoate, and diethyleneglycol diisononaoate; and esters of pentaerythritol such as pentaerythrityl tetraisostearate;

linear or branching hydrocarbons of mineral or synthetic origin, such as optionally volatile paraffin oils and derivatives thereof, Vaseline, polydecenes, hydrogenated polyisobutene, such as parleam oil;

fatty acids having 8 to 26 carbon atoms, such as cetyl alcohol, stearyl alcohol, and mixtures thereof (cetylstearyl alcohol), octyldodecanol, 2-butyloctanol, 2-hexyldecanol, 2-undecylpentadecanol, oleic alcohol, or linoleic alcohol;

fluorine-containing oils containing some hydrocarbons and/or silicones such as those described in document JP-A-2 295 912;

silicone oils such as optionally volatile polymethylsiloxanes (PDMS) having a linear or a cyclical silicon chain that are liquid or semiliquid at ambient temperature, in particular cyclopolydimethylsiloxanes (cyclomethicones) such as cyclohexasiloxane; polydimethylsiloxanes including alkyl, alcoxy, or phenyl groups, hanging from or at an end of the silicone chain, groups having 2 to 24 carbon atoms; phenyl silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes; and

mixtures thereof.

In various exemplary embodiments, compositions may also include at least one wax, at least one gum or at least one semi-solid fatty body of vegetal, animal, mineral or synthetic origin, optionally containing silicon.

Waxes may be hydrocarbon-containing, silicon-containing, and/or fluorine-containing, optionally including ester or hydroxyl functions. For example, waxes may be of natural origin.

Waxes may represent from 0.01% to 10% by weight or from 0.1% to 5% by weight relative to a total weight of a composition. Compositions may also be free from any wax.

In various exemplary embodiments, compositions may include at least one film-generating polymer. The term “film-generating polymer” refers to a polymer suitable, on its own or in the presence of an auxiliary film-forming agent, for forming a continuous film that adheres to skin.

In various exemplary embodiments, film-generating polymers may be at least one polymer selected from the group comprising:

hydrosoluble film-generating polymers;

aqueous dispersions of hydrodispersible film-generating polymer particles, also known as “latex”; in which case, the composition must include an aqueous phase;

liposoluble film-generating polymers;

lipodispersible film-generating polymers in the form of non-aqueous dispersions of polymer particles, preferably dispersions of polymer particles, where necessary stabilized on their surface by at least one stabilizing agent in one or more silicone and/or hydrocarbon oils; these non-aqueous dispersions also being referred to as NADs; and

mixtures thereof.

Aqueous dispersions of film-generating polymers suitable for use in the invention include, but are not limited to, acrylic dispersions sold under the following names Neocryl XK-90®, Neocryl A-1070®, Neocryl A-1090®, Neocryl BT-62®, Neocryl A-1079®, Neocryl A-523® by the supplier Avecia-Neoresins, Dow Latex 432® sold by the supplier Dow Chemical, Daitosol 5000 AD® sold by the supplier Daito Kasey Kogyo; or indeed aqueous dispersions of polyurethane sold under the name Neorez R-981®, Neorez R-974® by the supplier Avecia-Neoresins, Avalure UR-405®, Avalure UR-410®, Avalure UR-425®, Avalure UR-450®, Sancure 875®, Sancure 878®, Sancure 2060® sold by the supplier Goodrich, Impranil 850 sold by the supplier Bayer and Aquamere H-1511® sold by the supplier Hydromer.

As an aqueous dispersion of a film-generating polymer, mention may also be made, for example, of polymer dispersions that result from radical polymerization of one or more radical monomers within and/or partially on the surface of pre-existing particles of at least one polymer selected from the group constituted by polyurethanes, polyureas, polyesters, polyesteramides, and/or alkydes. These polymers are generally referred to as hybrid polymers.

As examples of hydrosoluble film-generating polymers, mention can be made, for example, of proteins such as proteins of vegetable origin, anionic, cationic, ampohoretic, or non-ionic polymers of chitin or of chitosan, cellulose polymers, acrylic polymers or copolymers, vinyl polymers, polymers of natural origin, optionally modified, and mixtures thereof.

Dispersions of film-generating polymers in a liquid fatty phase, in the presence of stabilizing agents, are described in particular in EP-A-0 749 746, EPA-0 923 928 and EP-A-0 930 060.

As examples of liposoluble polymers, mention can be made, for example, of copolymers of vinyl ester (the vinyl group being directly bonded to the oxygen atom of the ester group and the vinyl ester having a saturated, linear or branching hydrocarbon radical with 1 to 19 carbon atoms bonded to the carbonyl of the ester group) and at least one other monomer which may be a vinyl ester (different from the already-present vinyl ester), an α-olefin (having 8 to 28 carbon atoms), an alkylvinylether (in which the alkyl group has 2 to 18 carbon atoms), or an allyl or a methallyl ester (having a linear or branching saturated hydrocarbon radical with 1 to 19 carbon atoms, bonded to the carbonyl of the ester group).

Film-generating polymers may be present in compositions, for example at a dry matter content of from 0.01% to 20% by weight or from 0.5% to 10% by weight relative to a total weight of a composition.

Compositions may also contain additives of the kind that are usual in cosmetics, such as emulsifiers, hydrophilic or lipophilic gelling agents, hyprophilic or lipophilic active agents, preservatives, antioxidants, solvents, fragrances, odor-absorbers and pH adjusters.

Quantities of these various additives are those that are conventionally used in the art of cosmetics, representing, for example, from 0.01% to 20% of a total weight of a composition. Depending on their natures, additives may be introduced into a fatty phase or into an aqueous phase.

In any event, additives, and quantities thereof, should be selected so as to avoid harming desired optical properties.

Wetting agents may be selected from anionic or non-anionic wetting agents. Reference can be made for example to the document “Encyclopedia of Chemical Technology, Kirk-Othmer”, 3rd ed., 22:333-432, (1979), for a definition of properties and functions of wetting agents, and for example at pp. 347-377 of “Kirk Othmer” for anionic and non-anionic wetting agents.

Wetting agents used may be selected, for example:

from non-anionic wetting agents: fatty acids, fatty alcohols, polyethoxyl or polyglycerol fatty alcohols such as polyethoxyl stearyl or cetylstearyl alcohols, esters of fatty acid and sacchorose, alkyl glucose esters, in particular polyoxyethylenated fatty esters of C₁-C₆ alkyl glucose and mixtures thereof; and

from anionic wetting agents: C₁₆-C₃₀ fatty acids neutralized by amines, ammonia, or alkali salts, and mixtures thereof.

As emulsifiers and co-emulsifiers, mention can be made, for example, of oil-in-water (O/W) emulsifiers such as fatty acid esters and polyethylene glycol, such as PEG-100 stearate, and fatty acid and glycerin esters such as glyceryl stearate, and also water-in-oil (W/O) emulsifiers such as those available under the trade names ABIL WE09, ABIL EM90, and ABIL EM97 from the supplier Degussa Goldschmidt, or mixtures of ethylene glycol acetyl stearate and glyceryl tristearate sold by the supplier Guardian under the trade name UNITWIX.

As hydrophilic gelling agents, mention can be made, for example, of carboxyvinyl polymers (carbomer), acrylic copolymers such as copolymers of acrylates and alkylacrylates, polyacrylamides, polysaccharides, natural gums, and clays, and as lipophilic gelling agents, mention can be made of modified clays such as bentones, metallic salts of fatty acids, hydrophobic silica, and polyethylenes.

As active agents that are commonplace in the field of cosmetics or skin care and that are suitable for being used, mention can be made in particular of all the active agents known for their activity on skin aging, such as keratolytic or peeling agents, e.g., α-hydroxy acids, β-hydroxy acids, α-ceto acids, β-ceto acids, retinoids and esters thereof, retinol, retinoic acid and derivatives thereof. Mention can also be made, for example, of extracts from veinotonic plants such as extracts of knee-holly and/or horse chestnut; xanthic bases such as caffeine; vitamins, such as for example vitamins A, B3, PP, B5, E, K1, and/or C, and derivatives of such vitamins, and in particular esters thereof; agents against free radicals; sun filters; moisturizers such as polyols; ceramides; DHEA and derivatives thereof; the Q10 coenzyme; bleaching and depigmenting agents such as kojic acid, extracts of scutellaria, mulberry, licorice, and/or camomile; derivatives of para-aminophenols, of arbutin, and derivatives and mixtures thereof.

For use in cosmetically treating fatty or medium skins, compositions according to the present invention may contain at least one active agent selected, for example, from: vitamins B3 and B5; zinc salts, in particular zinc oxide and zinc gluconate; salicylic acid and derivatives thereof such as n-octanoyl-5-salicylic acid; triclosan; capryloylglycine; a clove extract; octopirox; hexamidine; and azelaic acid and derivatives thereof.

It is also possible to include UVA and/or UVB filters in the composition, selected from organic filters and mineral filters, possibly coated in order to make them hydrophobic.

Exemplary compositions according to this invention may be presented in any dosage form of the kind normally used in cosmetics, and in particular may be in the form of an optionally-gelled oily solution, an emulsion obtained by dispersing a fatty phase in an aqueous phase (O/W), or vice versa (W/O), a triple emulsion (W/O/W or O/W/O), a vesicular dispersion of ionic type (liposomes or oleosomes) and/or of non-ionic type (niosomes), and/or a dispersion of nanocapsules or nanospheres.

Exemplary compositions according to this invention may be more or less fluid and may have the appearance of a cream, an ointment, a milk, a lotion, a serum, a paste, a foam or a gel. Exemplary compositions may also be in solid form, in particular in the form of a stick or a cup or in some other dosage form, for example in powder form. Optionally, compositions may have a consistency that is adapted to being applied by spraying.

In various exemplary embodiments, compositions may include foundation makeup having a soft focus effect in accordance with the invention.

Various exemplary embodiments include the use of compositions having a soft focus effect in accordance with the invention for making up skin, mucous membranes or nails.

This invention is illustrated by the following examples, which are merely for the purpose of illustration, and percentages are expressed throughout by weight relative to the total weight of the composition.

### EXAMPLE 1

TABLE 2

| | weight % |
|---|---|
| Polygylceryl-4 isostearate (and) hexyl | 9 |
| (and) cetyl PEG/PPG-10/1 | |
| dimethicone | |
| Glycol acetyl stearate | 0.7 |
| Butyl p-hydroxybenzoate | 0.15 |
| 2-oleamido-1,3-octadecanediol | 0.04 |
| Cyclopentasiloxane (and) | 8 |
| disteardimonium hectoriate (and) | |
| denatured alcohol | |
| Cyclopentasiloxane | 14.25 |
| Dimethicone | 4 |
| Isododecane | 2.6 |
| Cloverleaf NS pigment from | 3 |
| Chemicals and Catalysts | |
| Talc P3 from Nippon Talc | 8 |
| Water | 44.06 |
| Magnesium sulfate | 0.7 |
| Methyl p-hydroxybenzoate | 0.25 |
| Glycol propylene | 5 |
| Chlorphenesine | 0.25 |

The following values were measured:

Th≧90%

H≧75%

C*≈20

### EXAMPLE 2

TABLE 3

| | weight % |
|---|---|
| Polygylceryl-4 isostearate (and) hexyl | 9 |
| (and) cetyl PEG/PPG-10/1 | |
| dimethicone | |
| Glycol acetyl stearate | 0.7 |
| Butyl p-hydroxybenzoate | 0.15 |
| 2-oleamido-1,3-octadecanediol | 0.04 |
| Cyclopentasiloxane (and) | 8 |
| disteardimonium hectoriate (and) | |
| denatured alcohol | |
| Cyclopentasiloxane | 14.25 |
| Dimethicone | 4 |
| Isododecane | 2.6 |
| Iron oxides (and) stearoyl disodium | 0.18 |
| glutamate (and) aluminum hydroxide | |
| Titanium dioxide (and) stearoyl | 0.32 |
| disodium glutamate (and) aluminum | |
| hydroxide | |
| Talc | 8 |
| Water | 46.56 |
| Magnesium sulfate | 0.7 |
| Methyl p-hydroxybenzoate | 0.25 |
| Glycol propylene | 5 |
| Chlorphenesine | 0.25 |

The following values were measured:

Th≧80%

H≧80%

C*≈25

It is observed that these two compositions enable skin defects to be attenuated, while also providing color and conferring natural-looking makeup.

The contrast between dark zones and light zones is reduced.

While this invention has been described in conjunction with the exemplary embodiments and examples outlined above, various alternatives, modifications, variations, improvements and/or substantial equivalents, whether known or that are or may be presently unforeseen, may become apparent to those having at least ordinary skill in the art. Accordingly, the exemplary embodiments of the invention, as set forth above, are intended to be illustrative, not limiting. Various changes may be made without departing from the spirit and scope of the invention. Therefore, the invention is intended to embrace all known or later developed alternatives, modifications, variations, improvements and/or substantial equivalents.

What is claimed is:

1. A colored cosmetic composition, comprising:
a filler and a coloring agent in a physiologically acceptable medium;
the coloring agent having a transparency and being present in a quantity such that presence of the coloring agent with the filler in the composition results in the composition having a saturation C* of at least 17, a hemispherical transmittance of at least 70%, and a soft focus index H of at least 40%.

2. The composition according to claim 1, wherein the hemispherical transmittance Th is from 80% to 100%.

3. The composition according to claim 1, wherein the filler is selected from the group consisting of powders of silica and silicates, powders of polymethylmethacrylate, talc, silica/TiO₂ or silica/zinc oxide composites, polyethylene powders, starch powders, polyamide powders, styrene/acrylic copolymer powders, silicone elastomers and mixtures thereof.

4. The composition according to claim 1, wherein the filler comprises particles having a number mean size of 15 μm or less.

5. The composition according to claim 1, wherein the filler is present in an amount of from 0.1% to 20% by weight relative to a total weight of the composition.

6. The composition according to claim 1, wherein the filler is present in an amount of about 8% by weight relative to a total weight of the composition.

7. The composition according to claim 1, wherein the coloring agent comprises particles of at least one pigment.

8. The composition according to claim 7, wherein the pigment is a non-interference pigment.

9. The composition according to claim 7, wherein the pigment is non-fluorescent.

10. The composition according to claim 7, wherein the pigment is present in an amount of at least 1% by weight relative to a total weight of the composition.

11. The composition according to claim 7, wherein the pigment has a contrast ratio of from 15 to 65.

12. The composition according to claim 11, wherein the contrast ratio of the pigment 50 or less.

13. The composition according to claim 11, wherein the contrast ratio is 40 or less.

14. The composition according to claim 10, wherein the pigment has a structure of a sericite/brown iron oxide/titanium dioxide/silica type.

15. The composition according to claim 10, wherein the pigment has a structure of a silica microsphere type containing yellow iron oxide.

16. The composition according to claim 7, wherein the composition comprises less than 2% by weight of pigment relative to a total weight of the composition.

17. The composition according to claim 7, wherein the composition comprises less than 1.5% by weight of pigment relative to a total weight of the composition.

18. The composition according to claim 7, wherein the composition comprises less than 1% by weight of pigment relative to a total weight of the composition.

19. The composition according to claim 7, wherein the composition comprises less than 0.7% by weight of pigment relative to a total weight of the composition.

20. The composition according to claim 7, wherein the composition comprises from 0.25% to 0.75% of the pigment relative to a total weight of the composition.

21. The composition according to claim 20, wherein the composition comprises about 0.5% of the pigment relative to a total weight of the composition.

22. The composition according to claim 1, wherein the coloring agent comprises particles of at least one of iron oxide and titanium dioxide type pigments.

23. The composition according to claim 1, wherein the saturation C* is 20 or more.

24. The composition according to claim 1, wherein the saturation C* is 60 or less.

25. A foundation makeup comprising the composition according to claim 1.
